(19) United States
(12) Patent Application Publication
(10) Pub. No.: US 2026/0051378 A1
(43) Pub. Date: Feb. 19, 2026

(54) HYBRID DATA SYNCHRONIZER

(71) Applicant: VMC MAR COM Inc., Knoxville, TN (US)

(72) Inventor: Mohammadreza Nehzati, Berlin (DE)

(21) Appl. No.: 19/304,277

(22) Filed: Aug. 19, 2025

Related U.S. Application Data
(60) Provisional application No. 63/684,539, filed on Aug. 19, 2024.

(51) Int. Cl. G16H 10/60; G06F 16/21; G06F 16/27

(52) U.S. Cl. CPC G16H 10/60; G06F 16/212; G06F 16/27

(57) ABSTRACT

Embodiments of the present disclosure relate to synchronizing and managing data. A first event is received from a data source. The first event comprises an envelope comprising schema information associated with the first event. Schema drift is detected based at least in part on the schema information. A signal indicative of the schema drift is emitted to a mapping module. An activation ticket is received from the mapping module. The activation ticket corresponds to an updated mapping profile. The updated mapping profile is based at least in part on the signal. A second event is received from the data source. The second event is bound with the updated mapping profile.

### RELATED PATENT APPLICATIONS

This patent application claims the benefit of U.S. Provisional Patent Application No. 63/684,539, filed on Aug. 19, 2024. The entire content of the foregoing patent application is incorporated herein by reference, including all text, tables and drawings.

### BACKGROUND

This disclosure generally relates to synchronizing data in software systems, such as systems for managing and/or analyzing electronic health records (EHRs).

In modern computing environments, organizations increasingly rely on a combination of on-premises databases, cloud-based application programming interfaces (APIs), and applications to store and exchange critical data. Synchronizing data across these heterogeneous systems presents numerous challenges, including differences in schema structure, update frequency, and access methods. Moreover, ensuring bidirectional consistency, maintaining compliance with data contracts and privacy regulations, and preserving data integrity during schema drift or connectivity disruptions are persistent obstacles. These challenges are compounded in multi-tenant or distributed deployments, where failures in flow control, auditing, or policy enforcement can result in data loss, corruption, or unauthorized exposure.

### SUMMARY

Traditional data integration and synchronization solutions typically focus on either batch-oriented extract-transform-load (ETL) pipelines or narrow real-time connectors tailored to specific platforms. Such approaches often lack resilience to schema drift (a condition in which the schema of a data source evolves over time such as by the addition, removal, or modification of fields, creating inconsistencies that may be detected and managed to maintain synchronization integrity), provide limited support for bi-directional writes, and fail to operate consistently across diverse environments that include on-premises databases, cloud APIs, and UI-only systems. Moreover, conventional solutions frequently treat security, compliance, and auditability as add-on features rather than foundational elements, resulting in brittle architectures that are difficult to adapt to evolving regulatory and operational requirements.

The present invention provides, in some embodiments, a hybrid data synchronizer capable of acquiring, signaling, and safely writing data across heterogeneous systems through multiple acquisition channels, including on-premises databases, cloud APIs, and agentic UI-driven interfaces. The system may be configured to detect and signal schema or distribution changes in real time, enforce data contracts at publish time, and manage profile activation through cryptographically signed activation tickets. This architecture allows mapping intelligence systems to test and validate new profiles before full activation, thereby preserving data integrity during change events.

Some embodiments include modules for flow control, security, observability, writeback transaction safety, multi-tenancy, and offline deployment, each providing specialized capabilities. Flow control may isolate priority data lanes and prevent bulk traffic from delaying critical updates. Security features may enforce end-to-end encryption, consent scope tagging, and immutable attestation logs. Observability components may provide real-time metrics, traces, and runbooks to ensure operational transparency. Writeback mechanisms may guarantee idempotent and reversible updates, while multi-tenancy controls may isolate tenant workloads and attribute resource consumption. Edge deployment features may allow sites to continue operating securely while disconnected, with replay and reconciliation upon reconnection.

Accordingly, some embodiments provide a resilient, policy-compliant, and audit-ready synchronization fabric that functions as the “data bloodstream” of a distributed enterprise ecosystem.

### BRIEF DESCRIPTION OF THE DRAWINGS

The above-mentioned aspects and other aspects of the present techniques will be better understood when the present application is read in view of the following figures in which like numbers indicate similar or identical elements.

FIG. 1A is a logical-architecture block diagram that illustrates a system configured for training and inference via neural networks.

FIG. 1B illustrates a second potential embodiment of the system shown in FIG. 1A.

FIG. 1C illustrates a third potential embodiment of the system shown in FIGS. 1A and 1B.

FIG. 1D illustrates a fourth potential embodiment of the system shown in FIG. 1A, FIG. 1B, and FIG. 1C.

FIG. 2 illustrates an example schematic for an example data management system.

FIG. 3 illustrates an example sequence diagram for data management.

### DETAILED DESCRIPTION OF CERTAIN EMBODIMENTS

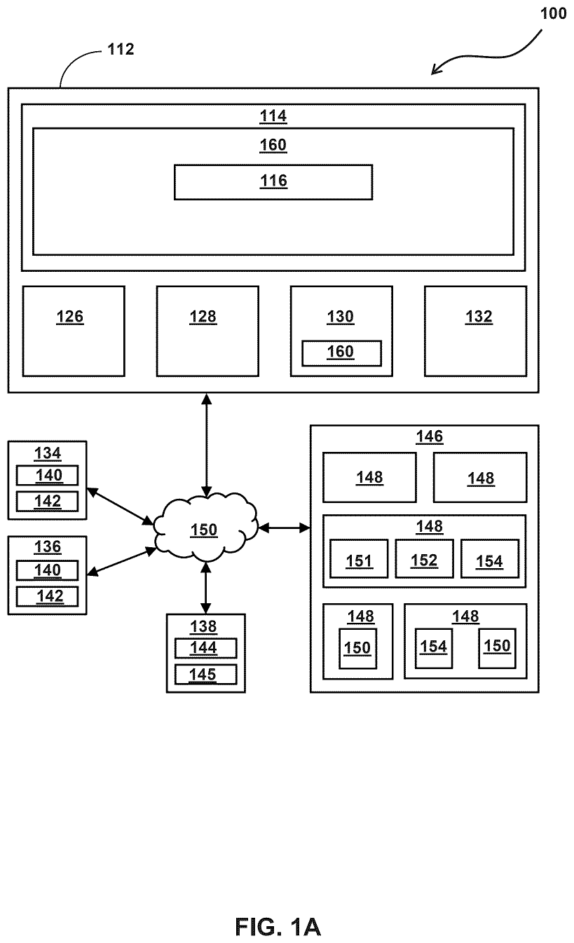

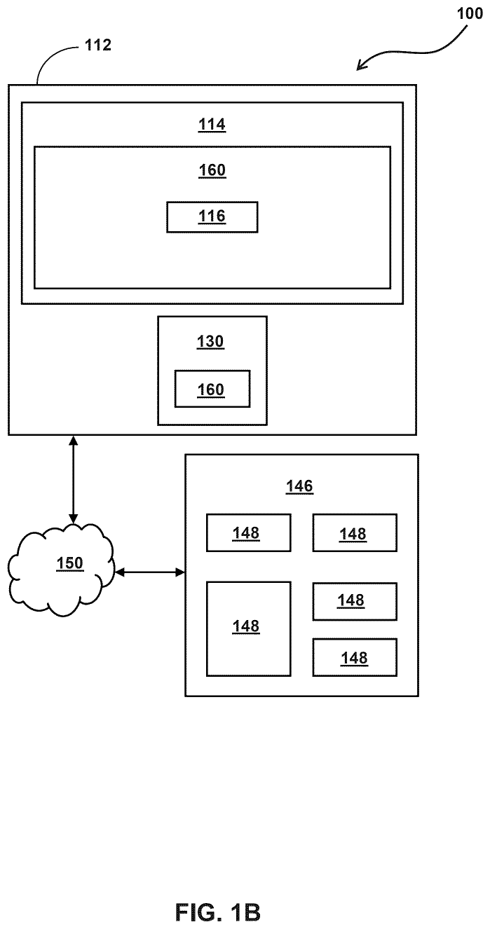

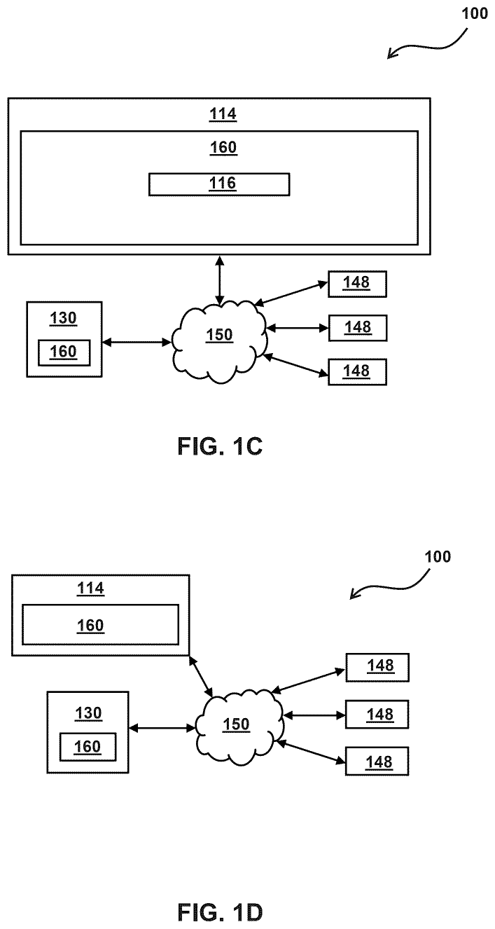

FIG. 1A illustrates a system 100 comprising a computing engine 112 and other components configured for data management. In embodiments, the system 100 provides a resilient, policy-compliant, and audit-ready synchronization fabric that functions as the “data bloodstream” of a distributed enterprise ecosystem.

Existing systems for data synchronization often suffer from fragmentation, inflexibility, and fragility when deployed across heterogeneous enterprise environments. Traditional extract-transform-load (ETL) pipelines are typically designed for batch processing, introducing latency that is unacceptable for applications requiring real-time or near real-time visibility. Meanwhile, purpose-built connectors for specific platforms or APIs may deliver lower latency but tend to be brittle, breaking when schemas evolve or when vendors adjust rate limits and authentication protocols. User interface (UI)-based systems are even more challenging, relying on fragile screen-scraping methods that often fail under modest changes in layout or authentication flows. Collectively, these conventional approaches do not adequately address the need for continuous, policy-governed, and auditable synchronization across on-premises databases, cloud services, and UI-driven applications.

A further shortcoming of prior systems is their lack of robust schema management and enforcement. Conventional synchronization technologies often treat schema drift as an afterthought, either failing silently or requiring manual intervention when data sources change. This leads to downstream inconsistencies, data corruption, or outages in dependent systems. Similarly, many existing systems lack mechanisms for controlled activation of new schema mappings, relying instead on immediate cutovers that risk disrupting production pipelines. The absence of contract enforcement at publish time further increases the likelihood of non-compliant or malformed data propagating across the ecosystem.

The system 100 addresses these shortcomings by introducing a hybrid data synchronizer configured to acquire data through multiple robust channels—including on-premises database change data capture (CDC), cloud and API integrations, and agentic UI automation—while unifying them under a resilient, policy-compliant architecture. In some embodiments, the system incorporates a schema signaling module that continuously detects schema changes, generates signed signals, and exposes replayable sample windows. Profile activation may be governed by cryptographically signed activation tickets, allowing mapping intelligence components to safely test new profiles in shadow or canary modes before full activation. Through these and additional features, the system 100 allows safe, incremental adaptation to evolving data sources without interrupting production flows.

Additionally, in some embodiments, the system 100 resolves longstanding challenges in operational resilience, governance, and compliance. A flow control module may provide backpressure handling, priority-lane routing, and disk-backed buffering to ensure continuity under heavy load or network disruption. A writeback module may guarantee safe, reversible, and idempotent outbound updates, enabling trusted bi-directional synchronization. Security modules may enforce encryption at rest and in transit, consent scope tagging, and immutable attestation logs for every operational event. The resulting system 100 is resistant to disruption, auditable, and compliant with organizational and regulatory policies.

Through these innovations, the invention provides not merely an incremental improvement but a re-architecture of synchronization itself. Instead of relying on brittle connectors or batch-oriented ETL, the hybrid data synchronizer establishes a durable, policy-governed, and trust-enhancing substrate for enterprise data operations. This technical approach ensures that synchronization can be maintained across diverse systems, even in the presence of schema drift, regulatory change, or connectivity challenges, thereby overcoming the core limitations of conventional solutions.

The system 100 provides technical solutions to the technical problems of data fragility, inconsistency, and non-compliance (among others) that arise when synchronizing information across heterogeneous computing environments. The disclosed system addresses these and other technical challenges through specific technical mechanisms, including schema signaling with cryptographically verifiable change notifications, activation tickets that enforce controlled profile cutovers, and flow control techniques such as priority-lane isolation and disk-backed spill buffers. These features, among other disclosed herein, operate at the system architecture and protocol levels to preserve data integrity, improve latency, and manage compliance even under adverse conditions such as outages or unanticipated schema changes.

Returning to FIG. 1A, more details related to the technical solution(s) provided by system 100 are described below, after introducing the components of system 100 and describing their operation. It should be noted, however, that not all embodiments necessarily provide all of the benefits outlined herein, and some embodiments provide all or a subset of these benefits or different benefits, as various engineering and cost tradeoffs are envisioned, which is not to imply that other descriptions are limiting.

System 100 includes computing engine 112, mobile user devices 134 and 136, a desktop user device 138, and external resources 146. Interaction with users or other entities occurs via a website or a native application viewed on a desktop user device 138, a mobile user device 134 or 136, or other components. In some embodiments, interaction occurs via a desktop user device 138 such as a desktop computer, a mobile website viewed on a smart phone, tablet, or other mobile user device 134 or 136, or via a special-purpose native application executing on a smart phone, tablet, or other mobile user device.

In some embodiments, computing engine 112 includes one or more of a processor 114, an application program interface (API) server 126, a web server 128, a memory 130, and a cache server 132. These components, in some embodiments, communicate with one another in order to provide the functionality of computing engine 112 described herein.

To illustrate an example of the environment in which computing engine 112 operates, FIG. 1A includes a number of components with which computing engine 112 communicates: mobile user devices 134 and 136; a desktop user device 138; and external resources 146. Each of these devices communicates with computing engine 112 via a network 150, such as the Internet or the Internet in combination with various other networks, like local area networks, cellular networks, Wi-Fi networks, or personal area networks.

Mobile user devices 134 and 136 comprise smart phones, tablets, gaming devices, or other hand-held networked computing devices having a display, a user input device (e.g., buttons, keys, voice recognition, or a single or multi-touch touchscreen), memory (such as a tangible, machine-readable, non-transitory memory), a network interface, a portable energy source (e.g., a battery), and a processor (a term which, as used herein, includes one or more processors) coupled to each of these components. The memory of mobile user devices 134 and 136 stores instructions that when executed by the associated processor provide an operating system and various applications, including a web browser 142, a native mobile application 140, or both. The desktop user device 138 also includes a web browser 144, a native application 145, or other electronic resources. In addition, desktop user device 138 includes a monitor; a keyboard; a mouse; memory; a processor; and a tangible, non-transitory, machine-readable memory storing instructions that when executed by the processor provide an operating system and the web browser 144 or the native application 145.

Native applications 140 and 145, and web browsers 142 and 144, in some embodiments, are operative to provide a graphical user interface associated with a user, for example, which communicates with computing engine 112 and facilitates user interaction with data from computing engine 112. In some embodiments, computing engine 112 is stored on or otherwise executed by user computing resources (e.g., a user computer, server, etc., such as mobile user devices 134 and 136, and desktop user device 138 associated with a user), servers external to the user, or in other locations. In some embodiments, computing engine 112 is be run as an application (e.g., an app such as native application 140) on a server, a user computer, or other devices.

External resources 146 include sources of information such as databases, websites, etc.; external entities participating with system 100; one or more servers outside of system 100; a network (e.g., the internet); electronic storage; equipment related to Wi-Fi technology; equipment related to Bluetooth® technology; data entry devices; or other resources. External resources 146 include available data sources 148. Available data sources 148 may comprise a large and varying set of data sources, with many different characteristics. In some embodiments, available data sources 148 comprise databases 151 (which themselves comprise storage technologies of various types—e.g., tabular data, graph data, embedding vectors, etc.—the approach is not restricted to just tabular data, such as Kusto tables), data tables 152, columns of data 154, documents, charts, images, video, sensor data, or other data.

In some embodiments, available data sources 148 include electronic health records (EHRs). An EHR may be a digital version of a patient's medical history, maintained by one or more healthcare providers, that includes comprehensive health information. EHR data may be derived from a medical doctor, orthodontist, dentist, and/or other medical provider. EHRs may comprise patient demographics, diagnoses, treatments, medications, allergies, laboratory results, test results, clinical notes, vitals, imaging/radiology reports, care plans, billing information, insurance information, appointment information, referral/specialist reports, and/or other relevant information.

Even though only a small number of available data sources 148 are shown in FIG. 1A, these may represent tens, hundreds, thousands, millions, or billions of different available data sources 148. In some embodiments, some or all of the different available data sources 148 are co-located (e.g., in a database server associated with a user), or individual available data sources 148 are located remotely from other data sources 148 (e.g., in different database servers associated with an organization and located across the world).

In some embodiments, some or all of the functionality attributed to external resources 146 is provided by resources included in system 100. External resources 146 are configured to communicate with computing engine 112, mobile user devices 134 and 136, desktop user device 138, or other components of system 100 via wired or wireless connections, via network 150 (e.g., a local area network and/or the internet), via cellular technology, via Wi-Fi technology, or via other resources.

Thus, computing engine 112, in some embodiments, operates in the illustrated environment by communicating with a number of different devices and transmitting instructions to various devices to communicate with one another. The number of illustrated external resources 146, desktop user devices 138, and mobile user devices 136 and 134 is selected for explanatory purposes only, and embodiments are not limited to the specific number of any such devices illustrated by FIG. 1A, which is not to imply that other descriptions are limiting.

Memory 130 stores instructions 160 that, when executed by processor 114, cause processor 114 to execute the various operations described herein. In some embodiments, memory 130 stores or is configured to access other data required for training and/or inference, or other information that otherwise allows system 100 to function as described herein. In some embodiments, memory 130 includes various types of data stores, including relational or non-relational databases; image, document, etc., collections; or programming instructions related to storage and execution of a related multimodal model (large language models, generative models, etc.) for example. In some embodiments, such components are formed in a single database, or are stored in separate data structures. In some embodiments, memory 130 comprises electronic storage media that electronically stores information. In some embodiments, the electronic storage media of memory 130 includes one or both of system storage that is provided integrally (i.e., substantially non-removable) with system 100 or other storage that is connectable (wirelessly or via a wired connection) to system 100 via, for example, a port, a drive, a network (e.g., the Internet), etc. In some embodiments, memory 130 is (in whole or in part) a separate component within system 100, or memory 130 is provided (in whole or in part) integrally with one or more other components of system 100 (e.g., processor 114). In some embodiments, memory 130 is located in a data center, in a server that is part of external resources 146, in a computing device 134, 136, or 138, or in other locations. In some embodiments, memory 130 includes one or more of optically readable storage media, magnetically readable storage media, electrical charge-based storage media (e.g., EPROM, RAM, etc.), solid-state storage media, or other electronically readable storage media. In some embodiments, memory 130 stores software algorithms, information determined by processor 114, information received (e.g., a user input query or prompt) via a graphical user interface displayed on computing devices 134, 136, or 138, information received from external resources 146 (e.g., training data from an available data source 148), or other information accessed by system 100 to function as described herein.

Processor 114 is configured to coordinate the operation of the other components of computing engine 112 to provide the functionality described herein. In some embodiments, processor 114 is formed by two or more processors, for example. As shown in FIG. 1A, in some embodiments, instructions 160 comprise a synchronizer module 116. Processor 114 is configured to direct the operation of module 116 by software; hardware; firmware; some combination of software, hardware, or firmware; machine-readable instructions; or other mechanisms for configuring processing capabilities.

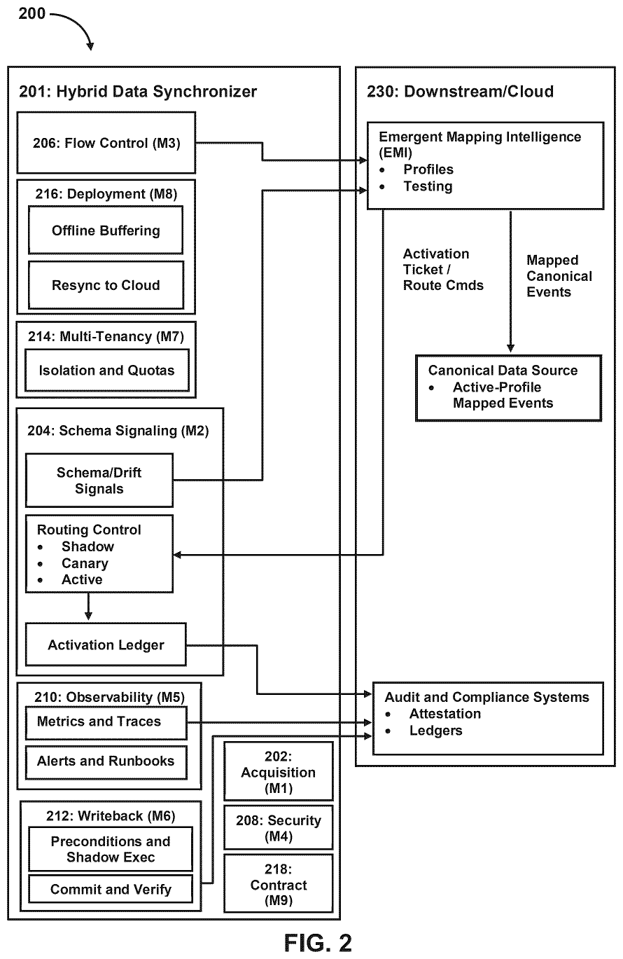

FIG. 2 is a schematic for an example data management system 200. As shown, the system 200 includes a synchronizer 201 (which may correspond to the synchronizer module 116 of FIG. 1) and downstream/cloud components 230. The synchronizer 201 may include an acquisition module 202, schema signaling module 204, flow control module 206, security module 208, observability module 210, writeback module 212, multi-tenancy module 214, deployment module 216, and/or contract module 218. In FIG. 2, solid lines may represent data flows; dashed lines may represent control signals.

Acquisition module 202 may be configured to establish and manage connectivity with one or more data sources. The data sources may contain EHRs. The acquisition module 202 may obtain data using multiple acquisition channels. The channels may be employed individually, in combination, or in a prioritized or failover sequence, without limitation, to provide resilient and policy-compliant access to structured and semi-structured data across diverse computing environments. The channels may include, without limitation: (i) an on-premises database channel; (ii) a cloud- or API-based channel; and/or (iii) an agentic user interface (UI) channel.

In one non-limiting embodiment, the on-premises database channel may support both initial data snapshot operations and change data capture (CDC) mechanisms. The snapshot operation may generate a baseline dataset by reading from one or more relational databases or equivalent structured storage systems. CDC may be performed using transaction log streaming, database-specific replication slots, or other incremental update capture techniques. In some cases, the on-premises database channel may further support optional writeback functionality, such as policy-gated, deterministic updates to one or more database records. The acquisition module 202 may operate in a full administrative mode, in which it is authorized to automatically discover available database endpoints within a local network, or in a scoped mode, in which explicit connection parameters are provided by a data owner or system integrator.

In another non-limiting embodiment, the cloud/API channel may acquire or deliver data through one or more standardized or proprietary network interfaces, including but not limited to REST, GraphQL, gRPC, or webhook-based APIs. The system may retrieve data using polling techniques with support for cursors, ETags, or timestamp-based deltas, and may additionally receive asynchronous notifications via webhooks or similar event-driven mechanisms. In various implementations, write operations over the cloud/API channel may be performed using mutation endpoints with precondition checks, such as version matching or ETag validation. These approaches may enhance data integrity and prevent conflicting updates.

In yet another non-limiting embodiment, the agentic UI channel may employ automated UI interaction to acquire and optionally update data from applications lacking direct database or API access. This channel may utilize headless browser automation tools in combination with machine vision and/or optical character recognition (OCR) models capable of interpreting the spatial and semantic layout of displayed content. Extracted data may be mapped to structured formats with confidence scoring. In some configurations, the acquisition module 202 may perform controlled UI-driven writebacks by filling and submitting forms programmatically under explicit authorization.

The acquisition module 202 may further include a connection readiness verification process applicable to any acquisition channel or combination thereof. This process may include, without limitation, performing authentication handshakes, schema or layout introspection, delta verification, write probe testing, and/or other pre-deployment checks. Upon successful completion of the readiness process, a given channel may be designated “connection ready” for operational use. Channels may be prioritized according to system policy or integrator configuration. The acquisition module 202 may automatically switch channels or fail over to alternative channels in response to connectivity loss, schema incompatibility, and/or policy-based ineligibility.

In various embodiments, configuration of connectivity parameters may be performed via an administrative interface or programmatic API. Such configuration may include source declarations with ownership attestations, consent scope specifications, channel-specific authentication credentials, and explicit read/write intent settings. Additional human verification switches may be configured to require operator approval before production data flows are initiated, and to gate writeback enablement on a per-entity basis. All channel selection and readiness determination activities may be logged with reason codes, enhancing operational auditability.

In certain embodiments, the system 200 includes a schema signaling module 204. The schema signaling module 204 may be configured to detect, communicate, and/or enforce changes in data source structure, semantics, and/or operational health. The schema signaling module 204 may operate in coordination with an external mapping intelligence system or equivalent analytical engine to ensure that data acquisition and routing are aligned with approved structural profiles. In various implementations, schema signaling module 204 may be adapted to function independently of downstream mapping logic, instead focusing on the identification and transmission of schema facts, the generation of change signals, and the controlled activation of mapping profiles in live data flows.

In one non-limiting embodiment, schema signaling module 204 may monitor (e.g., continuously monitor) incoming data streams for changes in schema, structure, or semantics. Such changes may include, without limitation, the addition, removal, or modification of fields; alterations in data types or enumerated values; adjustments to value ranges or units of measure; and variations in data distribution patterns. The module may generate signed, non-sensitive change and health signals referencing the relevant source, entity, and schema fingerprint, and may publish these signals to one or more downstream systems or message queues in near real time.

In some embodiments, schema signaling module 204 may maintain and distribute schema facts. Schema facts may characterize the structural state of a given source at a point in time. Schema facts may include a versioned schema snapshot linked to a registry entry, one or more schema fingerprints representing hash-based identifiers of structural characteristics, and references to active or historical data contract artifacts. The module may further expose sample and replay windows, which may comprise de-identified representative data slices (cold samples) or time-bounded event streams (hot windows) that can be replayed for testing and validation by an external mapping intelligence system.

In another non-limiting embodiment, schema signaling module 204 may enforce the activation of mapping profiles through a profile activation ticket mechanism. A mapping profile is a versioned configuration artifact that specifies how fields from a source schema are transformed, aligned, and bound to a canonical or target schema, and which may be activated using an activation ticket. An activation ticket may comprise a compact, cryptographically signed artifact issued by the external mapping intelligence system after validation and approval of a proposed mapping profile. Such a ticket may specify, without limitation: a profile identifier and version, associated source reference, expected schema fingerprints, approval signatures, routing plan (e.g., shadow, canary, or full activation), and validity constraints. Schema signaling module 204 may validate the ticket's authenticity, confirm compliance with integrator policies, and, upon successful verification, switch the routing of live data streams to the approved profile while recording the event in an immutable activation ledger.

In certain embodiments, schema signaling module 204 may manage traffic routing modes. This process may support gradual or controlled activation of new profiles. In a shadow mode, the module may duplicate incoming events to a shadow processing path without affecting downstream consumers. In a canary mode, the module may selectively route a percentage of incoming events to the new mapping path for partial deployment and validation. In an active mode, the module may bind incoming events to the approved profile identifier. The module may revert routing to a prior profile or to an unbound state in response to detected schema incompatibilities, expired activation tickets, or policy-based rollback triggers.

In various embodiments, schema signaling module 204 may stamp each event with the profile binding in effect at the time of acquisition and associate that event with its corresponding schema fingerprint, data contract identifier, and/or activation ticket record. Such lineage records may be stored in a manner that allows reconstruction of the operational context for any historical event, including profile version, activation approvals, and routing decisions.

In certain embodiments, the system 200 may include a flow control module 206. The system 200 may experience varying load conditions, network disruptions, and component failures. Accordingly, purposes of the flow control module 206 may include maintaining operational stability, data integrity, and performance. At a high level, the flow control module 206 may provide adaptive traffic management, prioritized data handling, resource safeguarding, and automated recovery mechanisms to ensure continuous read and write availability without data loss.

In one non-limiting embodiment, flow control module 206 may implement backpressure management to regulate the rate at which data is ingested, processed, and published. Backpressure techniques may include, without limitation, dynamically adjusting batch sizes based on measured latency or queue depth, enforcing bounded in-memory queues to prevent heap exhaustion, and spilling excess data to encrypted disk buffers upon reaching configurable high-water marks. The module may further track end-to-end watermarks for each channel to monitor latency targets and trigger remediation when thresholds are exceeded.

In some embodiments, flow control module 206 may support priority-lane routing for data events. A priority lane may be reserved for latency-sensitive domains such as clinical alerts or other mission-critical updates, while a bulk lane may be assigned to lower-priority or large-volume transfers such as historical backfills. These lanes may operate in isolation such that congestion in the bulk lane does not impair timely delivery in the priority lane. Lane assignment may be dynamically adjusted based on policy, event metadata, and/or observed system conditions.

In some embodiments, flow control module 206 may enforce resource management safeguards across memory, CPU, and storage subsystems. Memory safeguards may include configurable heap guards that shed non-essential tasks when thresholds are breached, streaming compaction of small batches to reduce overhead, and the use of off-heap buffers for large payload processing to avoid garbage collection stalls. CPU controls may include connector sandboxing with per-process quotas and cooperative yield scheduling to prevent task starvation. Storage management may involve rolling spill buffer files with retention limits, optional compression to optimize disk utilization, and checksum-based integrity verification for all on-disk buffers.

Flow control module 206 may define failure domain isolation to limit the scope of operational incidents. In some configurations, each acquisition connector may run in its own supervised process context. This may allow for targeted restarts without affecting other channels. Network partition handling may include an egress-only mode to permit local buffering during upstream outages, with configurable maximum offline durations to avoid overconsumption of resources. Disaster recovery features may include cold standby agents, hot failover nodes with shared offsets, and recovery point and time objectives (RPO/RTO) defined on a per-site basis.

In certain embodiments, flow control module 206 may implement replay and recovery capabilities that ensure exactly-once delivery after failures. Event offsets may be dual-persisted locally and in a cloud store, allowing recovery from the last committed position. Replays may be operator-initiated with explicit time and entity filters and may operate in bulk lanes to avoid impacting real-time traffic. Data integrity during replay may be validated via hash chaining, with quarantining of any mismatched records pending operator approval.

In various implementations, the module may employ graceful degradation strategies—e.g., to preserve functions during resource strain. Such strategies may include temporarily pausing low-priority lanes, switching CDC feeds to snapshot-only mode under severe API quota limits, suspending unstable channels, and disabling writeback operations until conditions normalize.

In some embodiments, the flow control module 206 may provide high availability and disaster recovery features—e.g., to maintain continuous operation of the synchronizer even in the presence of failures. For example, multiple synchronizer agents may be deployed in active and standby roles, with leader election procedures used to promote a standby agent to active status if the current leader becomes unavailable. In further embodiments, synchronizer clusters located in different geographic regions may be linked together, allowing a secondary region to assume responsibility in the event of a regional outage. The flow control module 206 may also mirror snapshots of spill buffer data and offset positions to standby nodes, allowing processing to resume without data loss or duplication if failover occurs. After the primary node or cluster has been restored, failback to the original system may be carried out under operator control, allowing administrators to manage the timing and safety of the transition.

In any mode of operation, flow control module 206 may expose observability metrics and alerts relevant to resilience. Such metrics may include channel-specific lag times, priority-lane latency, resource usage percentages, spill buffer capacity, and connector restart frequencies. Alerts may be triggered for threshold breaches, persistent restarts, excessive quarantine growth, or other resilience-related anomalies.

In some embodiments, the system 200 may include a security module 208. The security module 208 may perform operations to maintain confidentiality, integrity, privacy, and regulatory compliance of data from its acquisition through its delivery to downstream systems. The security module 208 may operate in conjunction with connectivity, flow control, and writeback components to enforce security policies, consent boundaries, and auditability requirements, for example.

In one non-limiting embodiment, security module 208 may implement authentication and authorization controls at multiple operational layers. Each acquisition connector may be provisioned with a distinct service principal per operation type (e.g., snapshot, change data capture, writeback), scoped to a single data source and granted the minimum privileges necessary to perform its function. Operator access to system commands, such as initiating a connection, executing a replay, or enabling writeback, may be controlled via role-based access control (RBAC) and optionally supplemented with attribute-based access control (ABAC) tied to factors such as tenant identity, site location, or jurisdiction. Multi-factor authentication (MFA) may be required for high-risk actions, such as schema contract overrides or profile activation co-signatures.

In some embodiments, security module 208 may provide credential and key management through integration with a hardware security module (HSM)-backed vault or equivalent secure storage system. Credentials may be short-lived, automatically rotated based on configurable time-to-live parameters, and isolated by channel or operational role. Cryptographic key separation may be maintained between signing, encryption, and authentication functions. Mutual TLS (mTLS) authentication may be enforced for inter-node and node-to-cloud communications, with proactive rotation and expiration alerts for certificates.

In another non-limiting embodiment, security module 208 may encrypt data in transit and/or at rest. Transport-layer encryption may be provided using TLS 1.3 or a higher available standard, while local spill buffers, state stores, and any other at-rest data repositories may be encrypted using AES-256-GCM or a functionally equivalent algorithm. Data integrity may be verified through envelope-level hashing, wherein each event payload may be cryptographically hashed and linked to the previous event hash, thereby establishing an immutable chain of custody. In addition, each event envelope may be digitally signed using Ed25519, ECDSA, or equivalent cryptographic algorithms to confirm authenticity and detect tampering.

Security module 208 may further provide privacy and data minimization mechanisms at the point of acquisition. These may include irreversible tokenization for analytics-scope data, reversible tokenization with key escrow for treatment-scope data, and configurable field-level redaction rules. The module may enforce consent scope tagging for each event, rejecting or quarantining events that are routed to a destination not approved for the associated consent scope.

In certain embodiments, security module 208 may support regulatory compliance alignment with frameworks such as HIPAA, GDPR, and/or SOC 2. HIPAA compliance features may include designation as a “conduit” for protected health information (PHI) and retention of audit logs for regulatory periods, while GDPR support may include mechanisms for right-to-erasure requests and purpose-limitation enforcement via consent scope tags. SOC 2 controls may be implemented through change management logging, operator access auditing, and/or integrity verification of configuration files.

The security module 208 may additionally maintain immutable attestation logs recording all operational events, including connection establishment, activation, writeback, replay, and/or schema overrides. Each log entry may include a sanitized parameter set, timestamp, actor identity, policy version, and digital signature. On-demand compliance reports may be generated to enumerate active and historical connections, credential rotation history, consent scope usage statistics, and event integrity verification results. The module may also provide full lineage traceability for downstream records, linking the record to its source, acquisition channel, data contract artifact, activation ticket, and original envelope signature.

In some implementations, the security module 208 may perform security operations functions, including anomaly detection for unusual connection patterns, schema changes outside approved change windows, or repeated writeback failures. Incident response hooks may allow immediate connector quarantine upon suspected credential compromise, as well as secure erasure of local buffers in response to breach triggers.

In certain embodiments, the system 200 includes an observability module 210. The observability module 210 may provide visibility into the health, performance, and/or data flows of the system 200. The observability module 210 may emit structured metrics, traces, and logs; enforce service-level objectives (SLOs) and error budgets; and/or define operational runbooks for incident response.

In some embodiments, observability module 210 generates metrics describing the operational state of acquisition, processing, and publishing pipelines. Such metrics may include, without limitation: ingestion throughput (e.g., events per second per channel), latency distributions (e.g., average, P95, and P99 times from source to publish), change data capture (CDC) lag in seconds, in-memory and/or spill buffer utilization percentages, connector uptime, connector restart counts, heartbeat freshness per source, credential age, consent scope distribution of events, and failover counts. Metrics may be emitted in standardized formats, such as Prometheus or OpenTelemetry.

In some embodiments, observability module 210 may produce distributed traces for individual data events. Each trace may link the lifecycle of an event across system stages, including source acquisition, envelope creation, intermediate buffering, publishing, and/or acknowledgment. Trace identifiers may be propagated downstream to mapping and transformation layers. This may allow end-to-end latency analysis and bottleneck identification. Slow path visualization may highlight delayed or stalled operations, such as elevated UI-channel fetch latency.

In some embodiments, observability module 210 may emit structured logs that include event identifiers, source identifiers, connector versions, operator actions, policy versions, and security context (e.g., signature identifiers, access role). Logs may be maintained in a JSON or equivalent structured format and stored according to configurable retention policies, such as short-term retention on local edge systems and long-term aggregation in a cloud logging service. Logs may support severity levels ranging from debug to fatal.

The observability module 210 may define and/or enforce service-level objectives (SLOs) and corresponding error budgets. Exemplary SLOs may include, without limitation: high-priority event latency (e.g., P95 less than five seconds from source to publish), CDC lag thresholds, zero tolerance for data loss, minimum connector uptime percentages, and bounded spill buffer usage. Error budgets may define acceptable deviations from SLOs over specified time windows, with violations triggering escalations or automated protective actions.

In certain embodiments, observability module 210 may support alerting rules and escalation workflows. Critical alerts may be raised for persistent latency breaches, prolonged connector downtime, CDC lag exceeding thresholds, spill buffer overutilization, heartbeat loss, and/or attestation verification failures. Warnings may be generated for precursor conditions such as high connector restart frequency, near-expiring credentials, or anomalies in tokenization. Alerts may be routed through operator dashboards, messaging systems, or paging services, and acknowledgments may be logged for auditability.

Observability module 210 may further provide operational runbooks for common failure modes, allowing automated or guided remediation by operators. Runbooks may include, without limitation: procedures for resolving CDC lag spikes (e.g., checking replication slot status, reprioritizing bulk lanes, adjusting batch sizes), actions for managing spill buffer saturation (e.g., increasing disk allocation, draining non-critical lanes), responses to connector crash loops (e.g., rollback to last stable version, quarantine of affected sources), and/or steps for addressing attestation or signature failures (e.g., quarantining events, reissuing certificates).

In some implementations, observability module 210 may provide dashboards tailored to system operators. Dashboards may include real-time throughput and latency panels, resilience heatmaps tracking failover events and latency histograms, security dashboards showing consent scope distributions and attestation pass/fail counts, and operational heatmaps highlighting connector instability and buffer usage trends.

In certain embodiments, the system 200 includes a writeback module 212. The writeback module 212 may execute outbound write operations to source systems. The writeback module 212 may apply deterministic safety mechanisms such as idempotency keys, precondition checks, rollback strategies, and immutable audit logging to ensure that all outbound updates are consistent with data contracts, consent scopes, and organizational policies.

In non-limiting embodiments, writeback module 212 may implement an idempotent writeback model, wherein outbound updates are uniquely identified by writeback event identifiers. The identifier may prevent duplication of operations and ensure that each update is applied at most once, regardless of retries, failovers, or network partitions. Writebacks may further be expressed as deterministic patches, specifying explicit field-level changes rather than opaque object replacements, thereby allowing precise verification of the intended modifications.

In some embodiments, writeback module 212 may apply precondition validation prior to committing any changes. Preconditions may include, without limitation: version or ETag matches, semantic guards preventing operations that violate domain-specific constraints (e.g., prohibiting deletion of active clinical records), and alignment with active data contract artifacts. Validation may include loading the current state of the target record, comparing it to expected conditions, and aborting the transaction if mismatches are detected.

In another non-limiting embodiment, writeback module 212 may conduct a shadow execution phase prior to committing changes. During shadow execution, the proposed update may be applied in a non-committing mode (e.g., database rollback transaction, API dry-run, or simulated UI form submission) to verify that the operation is acceptable and would yield the expected result. If the shadow execution confirms alignment with preconditions and expected postconditions, the writeback module 212 may proceed to commit the update using least-privilege credentials.

In certain implementations, writeback module 212 may prepare and execute rollback strategies for committed operations. If a postcondition mismatch is detected after a commit, and the change is reversible, an automated undo patch may be applied. For non-reversible or high-risk operations, the affected records may be quarantined pending human intervention. Rollback events may be logged alongside the original operation, with flags indicating reversal status.

The writeback module 212 may further integrate human approval workflows—e.g., to enforce governance over high-risk or sensitive updates. Approval artifacts may be collected prior to commit, including cryptographic signatures and timestamps from authorized approvers. The system may verify the presence and validity of required approvals before permitting execution, and such approvals may be retained in immutable audit logs for compliance verification.

In some embodiments, writeback module 212 may support channel-specific writeback modes. For database channels, updates may be executed via parameterized stored procedures or prepared statements with optional row-level locks. For API channels, updates may leverage official PATCH or PUT endpoints with version-matching preconditions and retry policies honoring rate limits. For agentic UI channels, updates may involve programmatic form submissions captured during stable workflows, with post-update validation conducted via DOM inspection or API cross-checking.

The writeback module 212 may maintain immutable audit and compliance logs for writeback operations. Logs may include event metadata (e.g., writeback identifier, source, channel, entity), preconditions, postconditions, before- and after-state snapshots, outcome status (committed, rolled back, failed), actor and approver identities, and policy versions. These logs may be exportable to satisfy compliance frameworks such as HIPAA, GDPR, and SOC 2.

In certain embodiments, the system 200 includes a multi-tenancy module 214. The multi-tenancy module may be configured to enforce logical separation, fair allocation of resources, and predictable cost management across multiple tenants operating within a shared environment. The multi-tenancy module 214 may prevent workloads associated with one tenant from degrading the performance, data integrity, and/or policy compliance of other tenants, while also allowing granular quota enforcement and cost attribution.

In non-limiting embodiments, a tenant may be a logical grouping of data sources, acquisition channels, and/or associated configurations belonging to a single data owner. Tenants may be provisioned with independent authentication and authorization boundaries, tenant-specific data contract artifacts, and/or custom policy sets for approvals, writebacks, and consent enforcement. Multi-tenancy module 214 may enforce isolation mechanisms such as topic partitioning in event streams, process isolation for each tenant's connectors, segregated local state directories, and/or dedicated spill buffer paths, thereby preventing cross-access or cross-contamination between tenants.

In some embodiments, multi-tenancy module 214 may apply quota enforcement to regulate resource consumption at both ingestion and publishing stages. Quotas may be defined for throughput (e.g., maximum events per second), storage allocation (e.g., spill buffer size), compute usage (e.g., CPU time per connector process), and/or network bandwidth (e.g., maximum outbound traffic per tenant). When quotas are exceeded, the module may apply hard enforcement measures such as dropping events into a quarantine stream with violation reasons, or soft enforcement measures such as throttling through token bucket algorithms or adaptive backoff strategies.

In some embodiments, multi-tenancy module 214 may implement cost control policies to promote predictable operations and fair cost attribution. Tenant-specific budgets may be configured for activities such as API call volumes, database snapshot frequencies, or spill buffer retention durations. The module may schedule high-volume or costly operations during off-peak windows to reduce expense and system contention. Cost attribution may be supported by per-tenant metrics describing compute hours, storage usage, network consumption, and API calls by endpoint, which may be exported for billing or chargeback purposes.

In certain configurations, multi-tenancy module 214 may provide governance and fairness mechanisms—e.g., to balance shared infrastructure across tenants. Critical tenants may be granted priority classes or guaranteed minimum resource floors, while idle capacity may be temporarily redistributed to other tenants under a fair share scheduling policy. Preemption mechanisms may reclaim resources when high-priority tenants require them. Policy violations, such as unapproved schema changes, may be contained to the offending tenant's data flows without impacting others.

The multi-tenancy module 214 may additionally enforce security boundaries across tenants. All envelopes may be tagged with tenant identifiers, and cross-tenant message routing may be explicitly prohibited. Operator accounts may be scoped to a single tenant unless explicitly granted multi-tenant administrative rights. Audit logs may be segmented by tenant.

In some embodiments, multi-tenancy module 214 may expose tenant-specific observability features. Dashboards may be generated per tenant, showing throughput, latency, quota utilization, cost metrics, and quarantine events. Alerts may be triggered for quota breaches, spill buffer overutilization, or API budget exhaustion, allowing tenants and/or administrators to manage their allocations independently.

The system may further include a deployment module 216. The deployment module 216 may be configured to support operation of the system within edge environments, including on-premises and/or remote sites that may have limited or intermittent connectivity to cloud infrastructure. The deployment module 216 may permit autonomous site operation by maintaining local ingestion, applying policies and validations locally, buffering securely during disconnection, and/or resynchronizing with the cloud upon reconnection.

In one non-limiting embodiment, deployment module 216 may implement edge deployment patterns, which may be tailored to the available resources at a site. For higher-capacity environments, a local agent may be installed as an operating system-level service, configured with connectors for databases, APIs, and user interfaces, and authorized with tenant-specific credentials and policies retrieved from a cloud control plane. For resource-constrained environments, the module may support a lightweight mode in which only essential connectors are activated, with bulk or non-critical tasks deferred to the cloud when connectivity is restored.

In some embodiments, deployment module 216 may provide offline operation capabilities that preserve acquisition workflows during disconnection. The system may continue to ingest from all configured channels, apply consent tagging, enforce local data contract validation, and/or record immutable attestation logs for each event. Spill buffer storage may serve as the primary repository during offline operation, with configurable retention and rotation rules to prevent exhaustion of local resources. Compression techniques may be employed to maximize capacity while maintaining cryptographic integrity protections.

In some embodiments, deployment module 216 may implement connectivity loss detection and/or graceful degradation strategies. The deployment module 216 may monitor heartbeats with the cloud control plane, entering offline mode upon detecting prolonged communication failures. Under offline conditions, non-critical channels may be paused, while priority flows such as clinical alerts continue to be processed. Writeback operations may be suspended by default to prevent partial or conflicting updates, though critical, latency-sensitive writebacks may be permitted under explicit local configuration.

When connectivity is restored, deployment module 216 may conduct resynchronization procedure to safely reintegrate buffered data. Integrity verification may confirm the continuity of hash chains for locally stored events, and reconciliation with the last confirmed cloud offset may be performed. Buffered events may then be replayed in order, leveraging idempotency identifiers to prevent duplication. If schema changes occurred during the offline period, the module may emit schema change signals and quarantine impacted events until a new mapping profile is activated.

In certain embodiments, deployment module 216 may also expose local autonomy features that permit secure and compliant data usage during offline operation. These may include a local read API allowing on-site applications to query validated event streams, subject to consent scope and access controls, and a local writeback mode enabling latency-sensitive updates to be applied immediately, with deferred confirmation and reconciliation upon reconnection.

Deployment module 216 may further provide local observability and alerts—e.g., to inform operators of system health. Local dashboards may display buffer usage, event counts by channel, and/or quarantine activity. Alerts may be delivered through local channels and/or SMS to notify operators of conditions such as spill buffer nearing capacity, resource exhaustion, or prolonged offline states, for example.

The system 200 may include a contract module 218. The contract module 218 may be configured to declare, validate, and/or enforce data contracts and schema registry references—e.g., during the acquisition and publication lifecycle. The contract module 218 may ensure that published events conform to a formally approved specification.

In one non-limiting embodiment, contract module 218 may define a data contract artifact (DCA) as a versioned and cryptographically signed specification governing the permissible structure, semantics, and privacy handling of events. A DCA may include, without limitation: a contract identifier, version, source reference, linked schema registry identifiers, compatibility policies (e.g., backward or forward), semantic rules such as units or code systems, privacy directives including tokenization or redaction policies, consent scope rules, service-level objectives for freshness and completeness, and/or required approvals or change control procedures. Each DCA may be digitally signed by both the system and an authorized integrator.

In some embodiments, contract module 218 may manage the lifecycle of DCAs across states such as draft, review, active, deprecated, and/or retired. Draft contracts may be created through an API referencing a candidate schema, validated for syntactic correctness and privacy tags, and submitted for approval. Review states may require signatures from integrators, legal authorities, or compliance officers, after which contracts may be promoted to active state and enforced at publish time. Deprecated contracts may remain valid for a grace window to enable migration, and retired contracts may be fully rejected except for historical replay purposes. State transitions may be logged in an immutable contract ledger with timestamped signatures.

In another non-limiting embodiment, contract module 218 may enforce publish-time validation and compliance. For each event, the module may determine the applicable active contract, validate structural conformance against the referenced schema registry entry, verify semantic requirements such as allowable enumerations or units of measure, apply privacy transformations, enforce consent scope restrictions, and cryptographically sign the event with contract pointers. Non-conforming events may be quarantined and associated with violation signals indicating the reason for rejection.

In certain implementations, contract module 218 may integrate with a schema registry-supporting formats such as Avro, JSON Schema, and/or Protocol Buffers. Registry entries may define canonical schemas, enforce compatibility rules, and serve as authoritative references for each DCA version. The module may also cache schemas locally for edge operation, synchronizing with the registry upon reconnection. Schema ID pinning may prevent unauthorized schema drift, and only registry updates associated with approved DCA changes may be accepted.

The contract module 218 may further emit signals associated with contract lifecycle and enforcement. These may include signals for contract creation, activation, deprecation, and retirement, as well as violation signals for non-compliant events. Schema change detections from upstream sources may also be surfaced to mapping and governance layers.

In some embodiments, contract module 218 may support migration patterns—e.g., to minimize disruption during schema evolution. For additive changes considered backward compatible, the module may auto-accept the new schema under a new DCA version, allowing dual acceptance for a transition window. For breaking changes, new DCAs may be drafted and approved while older versions are marked deprecated with grace periods. Emergency hotfix DCAs may be allowed under low-risk classifications.

The module may also provide audit and reporting capabilities—e.g., to support compliance, governance, and forensic investigation. Reports may include contract usage histories, violation counts, time-to-resolution metrics, and/or lineage reconstruction linking events to the contract versions in force at the time of publication.

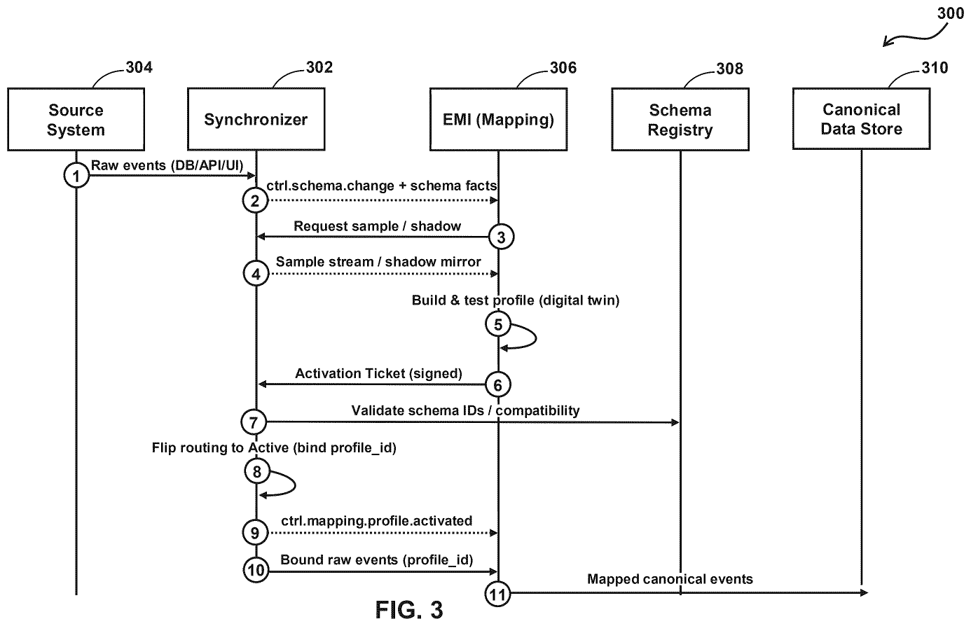

FIG. 3 is a non-limiting sequence diagram suitable for performing at least some functions described herein. FIG. 3 illustrates an example operational sequence where schema changes are detected, candidate mappings are tested under controlled routing, and a profile is securely activated through a ticket-based approval process. Although specific flows and message exchanges are depicted, the disclosure is not limited to the precise ordering or terminology shown; variations in sequencing, signaling, and control protocols may be implemented without departing from the present disclosure's scope.

The system 300 may include a synchronizer 302. The synchronizer 302 may perform any of the functions previously discussed with respect to the synchronizer 201 of FIG. 2. Additionally, as with FIG. 2, solid lines may represent data flows, and dashed lines may represent control signals.

As shown, a source system 304 may generate one or more raw events. An event is a discrete unit of data emitted by a source that represents an occurrence or state change, such as a record creation, update, or deletion, and that serves as the fundamental input processed and synchronized by the system. These events may be received by the synchronizer 302, which may acquire data through one or more connectivity channels. The synchronizer 302 may wrap the data in envelopes that include metadata, signatures, and/or consent scope information. The envelopes may be event envelopes, for example. An event envelope is a structured container that encapsulates raw event data together with associated metadata, digital signatures, and consent scope information. An event envelope may comprise schema information associated with an event. Schema information refers to the structural and semantic definitions describing the format, types, and permissible values of fields in a data source, which are used to validate, interpret, and map incoming events. The synchronizer 302 may emit schema change or health signals when relevant, as depicted by the early transmission of schema notifications to the emergent mapping intelligence (EMI) system 306.

The EMI system 306 may consume the schema signals, evaluate differences or drifts, and/or request sample data or replay windows for testing. As illustrated, the EMI system 306 may then issue a profile artifact that represents a candidate schema-to-canonical mapping configuration. This artifact may be conveyed back to the synchronizer 302 for staged evaluation. The synchronizer 302 may route a portion of the live data stream in shadow mode or canary mode to allow validation without disrupting production traffic.

The EMI system 306 may generate a signed activation ticket, which may include identifiers, versioning, schema fingerprints, and/or required approvals. As shown in FIG. 3, the activation ticket may be transmitted to the synchronizer 302, which may validate the signatures and policy compliance with schema registry 308 before switching routing modes. The synchronizer 302 may then bind live data streams to the approved profile, stamping events with the appropriate profile identifier and writing an immutable entry in an activation ledger.

Downstream consumers, such as a canonical data store 310 and audit or compliance systems, may then process only contract-compliant and profile-bound data events. The canonical store may receive data tagged with the active profile. Compliance systems may ingest the activation ledger entries and attestation logs.

Returning to FIG. 1A, system 100 can have many different forms, with or without some or all of the components shown in FIG. 1A, and still be configured to function as described. For example, FIG. 1B, FIG. 1C, and FIG. 1D illustrate examples of alternative potential embodiments of system 100. FIG. 1B illustrates system 100 without API server 126, web server 128, cache server 132, mobile user devices 134 and 136, or desktop user device 138 (e.g., which in this example are their own standalone devices, apart from system 100). FIG. 1C illustrates system 100 with processor 114, instructions 160 (including the module 116), memory 130 (which may or may not be included in the same computing structure as processor 114), and available data sources 148. In this example, the available data sources are each their own separate entity, not necessarily being related to each other. FIG. 1D illustrates system 100 with processor 114, instructions 160 (without being separately divided into the module 116), memory 130 (which again may or may not be included in the same computing structure as processor 114), and available data sources 148. Other embodiments with different arrangements of components are contemplated.

In FIGS. 1A-ID, the different components of system 100 are illustrated communicating via network 150. This is not intended to be limiting. As described herein, different components of system 100 communicate via network 150 (as shown), via wired connections, or via other wired or wireless connections. The illustrated components communicate directly with each other (e.g., via network 150 or a wired connection), or indirectly via other components of system 100.

Returning to FIG. 1A (and 1B, 1C, or 1D), it should be noted that in some embodiments, computing engine 112 is configured such that in the above-mentioned operations of processor 114, and input from users or sources of information inside or outside system 100, are processed by processor 114 through a variety of formats, including clicks, touches, uploads, downloads, etc. The illustrated components (e.g., processor 114, API server 126, web server 128, memory 130, and cache server 132) of computing engine 112 are depicted as discrete functional blocks, but embodiments are not limited to systems in which the functionality described herein is organized as illustrated by FIG. 1A. In some embodiments, the functionality provided by each of the components of computing engine 112 is provided by software or hardware modules that are differently organized than is presently depicted, for example such software or hardware is intermingled, broken up, distributed (e.g., within a data center or geographically), or otherwise differently organized. In some embodiments, the functionality described is provided by one or more processors of one or more computers executing code stored on a tangible, non-transitory, machine readable medium.

In some embodiments, computing engine 112 is provided with or within one or more portions of a multimodal model, or multiple multimodal models that include one or more neural networks. In some embodiments, these models, or portions thereof, are generated, executed, or otherwise utilized by computing engine 112 or processor 114 (or one or more of the components of processor 114) as shown in FIGS. 1A, 1B, and 1C, and described above.

In some embodiments, a multimodal model comprises a large language model (LLM), a generative model, or other models. In some embodiments, the multimodal model comprises one or more individual algorithms (e.g., that form a LLM, a generative model, a transformer, a neural network, an adapter, etc.). In some embodiments, an algorithm is a machine learning algorithm. In some embodiments, the machine learning algorithm is or includes a neural network, classification tree, decision tree, support vector machine, or other model that is trained and configured to output a response to input query. As an example, neural networks are based on a large collection of neural units (or artificial neurons). Neural networks loosely mimic the manner in which a biological brain works (e.g., via large clusters of biological neurons connected by axons). Each neural unit of a neural network is simulated as being connected with many other neural units of the neural network. Such connections can be enforcing or inhibitory in their effect on the activation state of connected neural units. In some embodiments, each individual neural unit has a summation function which combines the values of all its inputs together. In some embodiments, each connection (or the neural unit itself) has a threshold function such that the signal must surpass the threshold before it is allowed to propagate to other neural units. These neural network systems are self-learning and trained, rather than explicitly programmed, and can perform significantly better in certain areas of problem solving, as compared to traditional computer programs. In some embodiments, neural networks include multiple layers (e.g., where a signal path traverses from front layers to back layers). In some embodiments, back propagation techniques are utilized by the neural networks, where forward stimulation is used to reset weights on the “front” neural units. In some embodiments, stimulation and inhibition for neural networks is more free flowing, with connections interacting in a more chaotic and complex fashion.

Data is extracted by processor 114 or other components of system 100 from memory 130 or external resources 146, or other sources inside or outside system 100 in a secure and encrypted fashion. Data extraction by processor 114 is configured to be sufficient for system 100 to function as described herein, without compromising privacy or other requirements associated with a data source.

It should be appreciated that although module 116 are illustrated in FIGS. 1A (and 1B and 1C) as being co-located, one or more of module 116 may be located remotely from the other modules. The description of the functionality provided by the module 116 described below is for illustrative purposes, and is not intended to be limiting, as any module may provide more or less functionality than is described, which is not to imply that other descriptions are limiting. For example, one or more modules may be eliminated, and some or all of its functionality may be provided by other module(s), which is not to imply that other descriptions are limiting. As another example, processor 114 may be configured to control one or more additional modules that perform some or all of the functionality attributed to module 116.

Module 116 is program instructions that are executable by a processor 114 to implement one or more embodiments of the present techniques. In some embodiments, program instructions include a computer program (which in certain forms is known as a program, software, software application, script, or code). A computer program is written in a programming language, including compiled or interpreted languages, or declarative or procedural languages. In some embodiments, a computer program includes a unit suitable for use in a computing environment, including as a stand-alone program, a module, a component, or a subroutine. In some embodiments, a computer program corresponds to a file in a file system. A program is stored in a portion of a file that holds other programs or data (e.g., one or more scripts stored in a markup language document), in a single file dedicated to the program in question, or in multiple coordinated files (e.g., files that store one or more modules, sub programs, or portions of code). In some embodiments, a computer program is deployed to be executed on one or more computer processors located locally at one site or distributed across multiple remote sites and interconnected by a communication network, for example.

Cache server 132 expedites access to relevant data by storing likely relevant data in relatively high-speed memory, for example, in random-access memory or a solid-state drive (e.g., formed by at least a portion of memory 130). Web server 128 serves webpages having graphical user interfaces that display one or more views that facilitate receiving entry or selection of input from a user (e.g., including a query or command that system 100 perform a certain task, providing context, etc.), or other views. API server 126 serves data to various applications that process data related to user requested tasks, or other data. The operation of these components (API server 126, web server 128, and memory 130) is coordinated by processor 114, which bidirectionally communicates with each of these components or directs the components to communicate with one another. Communication occurs by transmitting data between separate computing devices (e.g., via transmission control protocol/internet protocol (TCP/IP) communication over a network), by transmitting data between separate applications or processes on one computing device; or by passing values to and from functions, modules, or objects within an application or process, e.g., by reference or by value.

API server 126 is configured to communicate user input query text commands, input images, and/or other information via a protocol, such as a representational-state-transfer (REST)-based API protocol over hypertext transfer protocol (HTTP) or other protocols. API requests identify which output data is to be determined, displayed, linked, modified, added, or retrieved by specifying criteria for identifying query intent tasks, such as queries for retrieving or processing information about a particular subject. In some embodiments, API server 126 communicates with native application 140 of the mobile user device 134, native application 145 of desktop user device 138, or other components of system 100.

Web server 128 is configured to display, link, modify, add, or retrieve portions or all of an output associated with a user input query, or other information encoded in a webpage (e.g. a collection of resources to be rendered by the browser and associated plug-ins, including execution of scripts, such as JavaScript™, invoked by the webpage). In some embodiments, the graphical user interface presented by the webpage includes inputs by which the user enters or selects data, such as clickable or touchable display regions or display regions for text input. For example, context information such as screen shots, documents, etc., may be uploaded, in combination with one or more entered text commands. Such inputs prompt the browser to request additional data from web server 128 or transmit data to web server 128, and web server 128 responds to such requests by obtaining the requested data and returning it to the user device or acting upon the transmitted data (e.g., storing posted data or executing posted commands). In some embodiments, the requests are for a new webpage or for data upon which client-side scripts will base changes in the webpage, such as XMLHttpRequest requests for data in a serialized format, e.g. JavaScript™ object notation (JSON) or extensible markup language (XML). Web server 128 communicates with web browsers, such as web browser 142 or 144 executed by user devices 136 or 138. In some embodiments, the webpage is modified by web server 128 based on the type of user device, e.g., with a mobile webpage having fewer and smaller images and a narrower width being presented to the mobile user device 136, and a larger, more content rich webpage being presented to the desktop user device 138. In some embodiments, an identifier of the type of user device, either mobile or non-mobile, for example, is encoded in the request for the webpage by the web browser (e.g., as a user agent type in an HTTP header associated with a GET request), and web server 128 selects the appropriate interface based on this embedded identifier, thereby providing an interface appropriately configured for the specific user device in use.

Web browsers 142 and 144 are configured to receive a website from computing engine 112 having data related to instructions (for example, instructions expressed in JavaScript™) that when executed by the browser (which is executed by the processor) cause mobile user devices 134 or 136, or desktop user device 138, to communicate with computing engine 112 and facilitate user interaction with data from computing engine 112. Native applications 140 and 145, and web browsers 142 and 144, upon rendering a webpage or a graphical user interface from computing engine 112, may generally be referred to as client applications of computing engine 112, which in some embodiments may be referred to as a server. Embodiments, however, are not limited to client/server architectures, and computing engine 112, as illustrated, may include a variety of components other than those functioning primarily as a server. Three user devices are shown, but embodiments are expected to interface with substantially more, with more than 100 concurrent sessions and serving more than 1 million users distributed over a relatively large geographic area, such as a state, the entire United States, and/or multiple countries across the world.

Though not illustrated in FIG. 1A (or 1B, 1C, or 1D), computing engine 112, in some embodiments, includes multiple processors 114, an input/output I/O device interface, and a network interface via an input/output (I/O) interface. In some embodiments, multiple processors are employed to provide for parallel or sequential execution of one or more portions of the techniques described herein. The I/O device interface provides an interface for connection of one or more I/O devices to computing engine 112. I/O devices include devices that receive input (e.g., from a user) or output information (e.g., to a user). I/O devices include, for example, graphical user interfaces presented on displays (e.g., a touchscreen or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices are connected to computing engine through a wired or wireless connection. I/O devices are connected to computing engine 112 from a remote location. I/O devices located on a remote computer system, for example, are connected to computing engine 112 via network 150 and the network interface.

The network interface includes a network adapter that provides for connection of computing engine 112 to network 150. The network interface facilitates data exchange between computing engine 112 and other devices connected to network 150. The network interface supports wired or wireless communication. In some embodiments, network 150 includes an electronic communication network, such as the Internet, a local area network (LAN), a wide area network (WAN), a cellular communications network, or the like.

The I/O interface is configured to coordinate I/O traffic between processors, memory 130, the network interface, I/O devices, or other peripheral devices. The I/O interface performs protocol, timing, or other data transformations to convert data signals from one component (e.g., memory 130) into a format suitable for use by another component (e.g., processor(s) 114). In some embodiments, the I/O interface includes support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing engine 112 or multiple computer systems configured to host different portions or instances of embodiments. Multiple computer systems may provide for parallel or sequential processing/execution of one or more portions of the techniques described herein.

While various items are illustrated as being stored in memory, these items or portions of them may be transferred between memory and other storage devices for purposes of memory management and data integrity. Alternatively, in other embodiments some or all of the software components executes in memory on another device and communicates with the illustrated computer system via inter-computer communication. In some embodiments, some or all of the system components or data structures are stored (e.g., as instructions or structured data) on a computer-accessible medium or a portable article to be read by an appropriate drive, various examples of which are described above. In some embodiments, instructions stored on a computer-accessible medium separate from computing engine 112 are transmitted to computing engine 112 via transmission media or signals such as electrical, electromagnetic, or digital signals, conveyed via a communication medium such as a network or a wireless link. Various embodiments include receiving, sending, or storing instructions or data implemented in accordance with the foregoing description upon a computer-accessible medium. Accordingly, the present techniques may be practiced with other computer system configurations.

To mitigate the problems described herein, the inventor had to both invent solutions and, in some cases just as importantly, recognize problems overlooked (or not yet foreseen) by others in the field of neural networks, and other fields. The inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium”. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several embodiments. Rather than separating those embodiments into multiple isolated patent applications, applicants have grouped these embodiments into a single document because their related subject matter lends itself to economies in the application process. But the distinct advantages and aspects of these embodiments should not be conflated. In some cases, embodiments address all of the deficiencies noted herein, but it should be understood that the embodiments are independently useful, and some embodiments address only a subset of such problems or offer other, unmentioned benefits that will be apparent to those of skill in the art reviewing the present disclosure. Due to cost constraints, some disclosed embodiments are not presently claimed and may be claimed in later filings, such as continuation applications or by amending the present claims. Similarly, due to space constraints, neither the Abstract nor the Summary sections of the present document should be taken as containing a comprehensive listing of all such embodiments or all aspects of such embodiments.

It should be understood that the description and the drawings are not intended to limit an embodiment to the particular form disclosed, but to the contrary, the intention is to cover all modifications, equivalents, and alternatives falling within the spirit and scope of the present embodiments as defined by the appended claims. Further modifications and alternative embodiments will be apparent to those skilled in the art in view of this description. Accordingly, this description and the drawings are to be construed as illustrative only and are for the purpose of teaching those skilled in the art the general manner of carrying out the embodiments. It is to be understood that the forms of the embodiments shown and described herein are to be taken as examples of embodiments. Elements and materials may be substituted for those illustrated and described herein, parts and processes may be reversed or omitted, and certain features may be utilized independently, all as would be apparent to one skilled in the art after having the benefit of this description. Changes may be made in the elements described without departing from the spirit and scope of the embodiments as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

1. A method comprising:
receiving a first event from a data source,
wherein the first event comprises an envelope comprising schema information associated with the first event;
detecting schema drift based at least in part on the schema information;
emitting, to a mapping module, a signal indicative of the schema drift;
receiving, from the mapping module, an activation ticket corresponding to an updated mapping profile, wherein the updated mapping profile is based at least in part on the signal;
receiving a second event from the data source; and
binding the second event with the updated mapping profile.

2. The method of claim 1, wherein the first event and the second event correspond to respective electronic health records (EHRs).

3. The method of claim 1, further comprising transmitting, to the mapping module, the second event bound with the updated mapping profile.

4. The method of claim 3, further comprising validating the second event via a schema registry.

5. The method of claim 4, wherein the second event is validated via the schema registry prior to transmitting the second event to the mapping module.

6. The method of claim 3, wherein the mapping module stores a mapped version of the second event in a canonical data store.

7. The method of claim 1, further comprising recording a record corresponding to the activation ticket in an activation ledger.

8. A system comprising:
a processor; and
memory storing instructions that, when executed by the processor, cause the processor to:
receive a first event from a data source,
wherein the first event comprises an envelope comprising schema information associated with the first event;
detect schema drift based at least in part on the schema information;
emit, to a mapping module, a signal indicative of the schema drift;
receive, from the mapping module, an activation ticket corresponding to an updated mapping profile, wherein the updated mapping profile is based at least in part on the signal;
receive a second event from the data source; and
bind the second event with the updated mapping profile.

9. The system of claim 8, wherein the first event and the second event correspond to respective electronic health records (EHRs).

10. The system of claim 8, wherein the instructions, when executed by the processor, further cause the processor to transmit, to the mapping module, the second event bound with the updated mapping profile.

11. The system of claim 10, wherein the instructions, when executed by the processor, further cause the processor to validate the second event via a schema registry.

12. The system of claim 11, wherein the second event is validated via the schema registry prior to transmitting the second event to the mapping module.

13. The system of claim 10, wherein the mapping module stores a mapped version of the second event in a canonical data store.

14. The system of claim 8, wherein the instructions, when executed by the processor, further cause the processor to record a record corresponding to the activation ticket in an activation ledger.

15. A non-transitory computer readable medium having instructions thereon, the instructions, when executed by a computer, causing the computer to perform operations comprising:
receiving a first event from a data source,
wherein the first event comprises an envelope comprising schema information associated with the first event;
detecting schema drift based at least in part on the schema information;
emitting, to a mapping module, a signal indicative of the schema drift;
receiving, from the mapping module, an activation ticket corresponding to an updated mapping profile, wherein the updated mapping profile is based at least in part on the signal;
receiving a second event from the data source; and
binding the second event with the updated mapping profile.

16. The medium of claim 15, wherein the first event and the second event correspond to respective electronic health records (EHRs).

17. The medium of claim 15, the operations further comprising transmitting, to the mapping module, the second event bound with the updated mapping profile.

18. The medium of claim 17, the operations further comprising validating the second event via a schema registry.

19. The medium of claim 18, wherein the second event is validated via the schema registry prior to transmitting the second event to the mapping module.

20. The medium of claim 17, wherein the mapping module stores a mapped version of the second event in a canonical data store.
